(19) United States
(12) Patent Application Publication
(10) Pub. No.: US 2009/0220593 A1
(43) Pub. Date: Sep. 3, 2009

(54) EXTENDED RELEASE DOSAGE FORMS OF QUETIAPINE

(76) Inventors: Inder GULATI, New Delhi (IN); Rajan Kumar Verma, New Delhi (IN); Rajeev Singh Raghuvanshi, Gurgaon (IN)

Correspondence Address: RANBAXY INC.;INTELLECTUAL PROPERTY DEPT., 600 COLLEGE ROAD EAST, SUITE 2100, PRINCETON, NJ, 08540, US

(21) Appl. No.: 12/361,248

(22) Filed: Jan. 28, 2009

(30) Foreign Application Priority Data
Jan. 29, 2008 (IN) 250/DEL/2008

(51) Int. Cl. A61K 9/58; A61K 31/554; A61K 9/22; A61K 9/14

(52) U.S. Cl. 424/462; 514/211.13; 424/468; 424/497

(57) ABSTRACT

The present invention relates to multiple unit extended release dosage forms of quetiapine for oral administration, wherein each unit comprises a core containing quetiapine and one or more of pharmaceutically acceptable excipients coated with a rate-controlling coating and process for the preparation thereof.

### FIELD OF THE INVENTION

The present invention relates to multiple unit extended release dosage forms for oral administration comprising quetiapine and the process for the preparation thereof.

### BACKGROUND OF THE INVENTION

Quetiapine is a psychotropic drug belonging to the chemical class of dibenzothiazepine derivatives and is chemically designated as 11-[4-[2-(2-hydroxyethoxy) ethyl]-1-piperazinyl]dibenzo[b,f][1,4]thiazepine. It is present in tablets as the hemifumarate salt. Quetiapine acts as an antagonist at several neurotransmitter receptors including dopamine D₁ and D₂ receptors, serotonin 5HTA_{A1} and 5HT₂ receptors, histamine H₁ receptor and adrenergic α₁ and α₂ receptors. Quetiapine is thought to exert its antipsychotic effects primarily via antagonism of dopamine D₂ receptor and serotonin 5HT₂ receptors.

Currently, quetiapine is commercially available as conventional immediate release tablets in 25, 50, 100, 200, 300 and 400 mg strengths marketed by Astra Zeneca, under the brand name Seroquel®, requiring two or three times a day dosing. It is also available as extended release tablets in 50, 200, 300 and 400 mg strengths under the brand name Seroquel® XR. These tablets contain lactose monohydrate, microcrystalline cellulose, sodium citrate, hypromellose, magnesium stearate and hypromellose, polyethylene glycol 400, titanium dioxide, yellow iron oxide (200 and 300 mg tablets) in the film coat.

It is desirable in the treatment of diseases both therapeutically and prophylactically to provide the active pharmaceutical ingredient in extended release form. Advantages of extended release systems over conventional are well known. Extended release dosage forms increase patient compliance due to reduction in frequency of dosing. They also reduce the severity and frequency of side effects, as they maintain substantially constant plasma levels. This is especially important in the treatment of schizophrenia and bipolar mania, for the alleviation of psychosis, where blood levels of medicament must be maintained at a therapeutically effective level to provide symptomatic relief.

Numerous systems have been devised for the purpose of obtaining extended release of the drug from the dosage form. One of the methods of achieving extended drug release involves the use of multiple unit systems. When multiple unit dosage forms are administered, the individual units are dispersed freely into the gastrointestinal contents, avoiding the high local concentration of drug which may lead to irritation of the gastrointestinal mucosa. The performance of such dosage forms is independent of inter and intra-patient variability in gastric emptying time because of the small size of the individual units. Multiple unit dosage forms possess large surface area, which promote complete and uniform absorption.

Quetiapine and its pharmaceutically acceptable salts, its preparation, physical properties and beneficial pharmacological properties are disclosed in U.S. Pat. No. 4,879,288 and European Patents 0 240 228 and 0 282 236.

A typical sustained release formulation of quetiapine is described in U.S. Pat. No. 5,948,437. It discloses matrix formulations of quetiapine wherein the matrix is comprised of gelling agents, particularly, hydroxypropylmethylcellulose for sustained release.

PCT application 2007/000778 exemplifies modified release matrix tablets comprising quetiapine fumarate, a polymer system in an amount of less than about 80% w/w of the composition comprising at least two swellable pH independent polymers wherein at least one is hydrophilic; and other pharmaceutically acceptable excipients.

PCT application 2007/110878 exemplifies hard gelatin capsules containing sustained release granules comprising quetiapine fumarate, at least one solubilizer (e.g., propylene glycol caprylate/caprate, Labrafac®), a release rate-controlling polymer system comprising hydrophilic polyethylene oxide and hydroxyethylcellulose, and other pharmaceutically acceptable excipients.

PCT application No. 2007/086079 discloses once-a-day sustained release matrix tablets comprising phenothiazine derivative, a channelizer, a rate controlling agent and suitable pharmaceutical excipients. Examples cited therein disclose sustained release formulations of quetiapine fumarate with the water-soluble polymer hydroxypropylmethylcellulose and other excipients.

U.S. Publication No. 2005/0158383 discloses sustained release dosage forms of quetiapine in a waxy matrix. In the formulations described therein, the coating composition comprising a hydrophilic polymer may be press coated onto the core.

PCT application No. 01/21179 discloses a granule formulation comprising quetiapine or a pharmaceutically acceptable salt thereof and freely or very water-soluble binder.

PCT application No. 03/39516 discloses a method for improving dissolution of poorly dispersible medicament like quetiapine, which comprises mixing the poorly dispersible medicament with a floating agent and/or a surfactant and granulating the mixture.

### SUMMARY OF THE INVENTION

Herein are disclosed multiple unit extended release dosage forms of quetiapine, wherein each unit comprises a core containing quetiapine and one or more of pharmaceutically acceptable excipients and a rate-controlling coating as well as process for the preparation thereof.

In one general aspect, multiple unit extended release dosage forms of quetiapine are provided, wherein each unit comprises a core containing quetiapine and one or more of pharmaceutically acceptable excipients and a rate-controlling coating.

In another general aspect, multiple unit extended release dosage forms of quetiapine are provided, wherein each unit comprises a core containing quetiapine and one or more of pharmaceutically acceptable excipients and a rate-controlling coating; wherein the dosage form provides therapeutically effective plasma levels of quetiapine for a period of upto about 24 hours.

In another general aspect, multiple unit extended release dosage forms of quetiapine are provided, wherein each unit comprises a core containing quetiapine and one or more of pharmaceutically acceptable excipients and a rate-controlling coating; wherein the core is an inert core coated with a solution/dispersion comprising quetiapine and one or more of pharmaceutically acceptable excipients.

In another general aspect, multiple unit extended release dosage forms of quetiapine are provided, wherein each unit comprises a core containing quetiapine and one or more of pharmaceutically acceptable excipients and a rate-controlling coating; wherein the core comprises quetiapine dispersed within it.

In another general aspect, multiple unit extended release dosage forms of quetiapine are provided, wherein each unit comprises a core containing quetiapine and one or more of pharmaceutically acceptable excipients and a rate-controlling coating; wherein the rate-controlling coating comprises water-soluble polymers or water-insoluble polymers.

In another general aspect, multiple unit extended release dosage forms of quetiapine are provided, wherein each unit comprises a core containing quetiapine and one or more of pharmaceutically acceptable excipients and a rate-controlling coating; wherein the rate-controlling coating comprises ammonio methacrylate copolymers and methacrylic acid copolymers.

In another general aspect, multiple unit extended release dosage forms of quetiapine are provided, wherein each unit comprises a core containing quetiapine and one or more of pharmaceutically acceptable excipients and a rate-controlling coating; wherein the rate-controlling coating comprises cellulose acetate.

In another general aspect, multiple unit extended release dosage forms of quetiapine are provided, wherein each unit comprises a core containing quetiapine and one or more of pharmaceutically acceptable excipients and a rate-controlling coating; wherein the rate-controlling coating comprises ethyl cellulose.

In another general aspect, multiple unit extended release dosage forms of quetiapine are provided, wherein each unit comprises a core containing quetiapine and one or more of pharmaceutically acceptable excipients and a rate-controlling coating; wherein the multiple units are compressed into tablets.

In another general aspect, multiple unit extended release dosage forms of quetiapine are provided, wherein each unit comprises a core containing quetiapine and one or more of pharmaceutically acceptable excipients and a rate-controlling coating; wherein the multiple units are filled into capsules or sachets.

In another general aspect, a process for the preparation of a multiple unit extended release dosage forms of quetiapine are provided, wherein the process comprises the steps of dispersing quetiapine and one or more of pharmaceutically acceptable excipients in a pharmaceutically acceptable solvent; layering the dispersed quetiapine on inert cores; and coating the drug layered cores with rate-controlling coating.

In another general aspect, a process for the preparation of a multiple unit extended release dosage forms of quetiapine are provided, wherein the process comprises the steps of mixing quetiapine with one or more of pharmaceutically acceptable excipients to form a drug core and coating the core with rate-controlling coating.

### DETAILED DESCRIPTION OF THE INVENTION

“Quetiapine”, as recited herein, means quetiapine or a pharmaceutically acceptable form of quetiapine, including without limitation, its free base form, and all pharmaceutically acceptable salts, complexes, enantiomer, solvates, hydrates, and polymorphs. An exemplary quetiapine salt is quetiapine hemifumarate.

The term “multiple unit extended release dosage form”, as used herein, includes multiplicity of individual coated units in the dosage form that achieves the slow release of drug over an extended period of time, and includes prolonged, controlled, extended and delayed release profiles. The multiple units may be designed as granules, pellets, compacts, beads, spheroids, and the like.

The term “inert core”, as used herein, includes water-insoluble, soluble and swellable cores. Water-insoluble inert cores include, for example, silicon dioxide, small particles of glass or plastic resin particles, e.g., polypropylene or polyethylene. Water-soluble inert cores include, for example, sugar spheres e.g. glucose, mannitol, lactose, xylitol, dextrose, sucrose, and salt cores, e.g. sodium chloride or potassium chloride. Water-swellable inert cores may be made up of microcrystalline cellulose (Celphere®), hydroxypropylmethylcellulose, starch or mixtures thereof.

The rate-controlling coating may comprise one or more of water-insoluble polymers or water-soluble polymers or mixtures thereof. The water-insoluble polymers may be selected from ammonio methacrylate copolymers e.g., Eudragit® RL and RS, and methacrylic acid copolymer e.g., Eudragit®L and S; cellulose acetate, ethylcellulose, polyethylene, high molecular weight polyvinyl alcohols. Water-soluble polymers may be selected from polyvinylpyrrolidone, hydroxypropylmethylcellulose, hydroxypropylcellulose, methylcellulose, polysaccharides (e.g., alginate, xanthan gum etc.), polyethylene oxide, methacrylic acid copolymers and derivatives or mixtures thereof.

The rate-controlling coating in addition to polymers may also comprise plasticizers such as triethyl citrate, tributyl citrate, dibutyl phthalate, diethyl phthalate, triacetin and polyethylene glycol or mixtures thereof.

A seal coat may be optionally, applied on the inert core which comprises one or more of the polymers selected from cellulose acetate, ethyl cellulose, hydroxypropylmethylcellulose, polyethylene glycol, polyvinylpyrrolidone and the like. The seal coat may be applied on the inert core prior to drug layering or between the drug layered cores and rate-controlling coating. Coated cores may be further coated with another coating layer. Such coatings prevents the rupturing or cracking of release-controlling layers or fragmentation of the core due to mechanical stress generated during compression of cores into tablets or filling into capsules/sachets. The coating layer may comprise polyethylene glycols or wax material, and the like.

The pharmaceutically acceptable excipients may be one or more of diluents, binders, disintegrants and glidants/lubricants. Suitable diluents may be selected from one or more of any conventional diluents such as microcrystalline cellulose, silicified microcrystalline cellulose, lactose, mannitol, sorbitol, calcium phosphate, calcium sulfate, calcium carbonate, starch, starch pregelatinized and the like. Suitable binders may be selected from one or more of polyvinylpyrrolidone, cross-linked polyvinylpyrrolidone, polyvinyl alcohol, carboxymethylcellulose, hydroxypropylmethylcellulose, hydroxypropylcellulose, microcrystalline cellulose, silicified microcrystalline cellulose, starch pregelatinized, and the like. Binders may also be present in drug layer to provide proper adhesion of the drug layer onto the inert core.

Suitable disintegrants may be selected from carboxymethylcellulose, sodium carboxymethylcellulose, cross-linked sodium carboxymethylcellulose, sodium starch glycolate, starch, pregelatinized starch, hydroxypropylstarch, and the like. Suitable glidants/lubricants may include one or more of magnesium stearate, calcium stearate, zinc stearate, stearic acid, talc, colloidal silicon dioxide, hydrogenated vegetable oil, polyethylene glycol, sodium stearyl fumarate, and the like. Suitable solvents employed for preparing solution/dispersion of polymers are selected from water, isopropyl alcohol, acetone, methanol, ethanol, dichloromethane or mixtures thereof.

The compositions described herein may be prepared by conventional processes using easily available excipients and conventional equipments. Coating solutions may be applied using conventional techniques such as spray coating in a coating pan or fluidized bed processing or dip coating, and the like.

Preferred pharmaceutical compositions of the present invention may take form of several different embodiments. In one embodiment, the multiple unit extended release dosage form of quetiapine comprises an inert core coated with a solution/dispersion of quetiapine and other pharmaceutically acceptable excipients. The drug layered cores are then coated with a rate-controlling coating. In the above embodiment, the inert core is coated with a seal coat comprising a solution/dispersion of cellulose acetate and diethyl phthalate in suitable solvents prior to drug layering.

In another embodiment, the multiple unit extended release dosage form of quetiapine comprises a core having quetiapine dispersed in it and a rate-controlling coating over the core.

In the above embodiments, the multiple unit dosage form of the invention may also include an inert coating on the core coated with quetiapine or on the core coated with rate-controlling layer. The multiple unit extended release dosage form of the above embodiments may also include an immediate release portion sufficient to provide initial therapeutic plasma levels. The immediate release portion may be the cores containing quetiapine devoid of the rate-controlling coating or quetiapine containing coating over the rate-controlling coating.

In another embodiment, the process for the preparation of multiple unit extended release dosage form of quetiapine comprise the steps of dispersing quetiapine and other pharmaceutically acceptable excipients in a solvent system and layering the drug solution/dispersion on the inert core. A seal coat may be optionally applied on the inert cores prior to drug layering. The drug layered cores are further coated with a rate-controlling coating.

The cores may be prepared by conventional techniques known in the art like granulation, extrusion and spheronization and quetiapine may be dispersed in the core or coated on the cores. The drug cores are then further coated with a rate-controlling coating. The core can be inert core readily available like non-pareil sugar beads or microcrystalline cellulose beads.

As a further process of the above embodiments, the coated cores may be compressed into tablets or filled into capsules or sachets. The tablets may further be film coated using conventional coating compositions like Opadry®.

In one particular embodiment, the process for the preparation of multiple unit extended release dosage form of quetiapine comprises the steps of:
a. loading the inert cores in the coating equipment;
b. optionally, coating the inert cores with a seal coat comprising a solution/dispersion of a suitable polymer;
c. applying a solution/dispersion of quetiapine and one or more of pharmaceutically acceptable excipients in a suitable solvent onto the inert cores;
d. applying a solution/dispersion of rate-controlling polymer and one or more of pharmaceutically acceptable excipients on the drug layered cores; and
e. optionally, applying a solution/dispersion of a suitable polymer in a suitable solvent on the extended release coated cores; and
f. blending the coated cores of step (e) with one or more of pharmaceutically acceptable excipients and then compressing into tablets.

In another particular embodiment, the process for the preparation of multiple unit extended release dosage form of quetiapine comprises the steps of:
a. loading the inert cores in the coating equipment;
b. optionally, coating the inert cores with a seal coat comprising a solution/dispersion of the polymer;
c. applying a solution/dispersion of quetiapine and one or more of pharmaceutically acceptable excipients in a suitable solvent onto the inert cores;
d. applying solution/dispersion of rate-controlling polymer and one or more of pharmaceutically acceptable excipients on the drug layered cores; and
e. filling the extended release coated pellets of step (d) in capsules or sachets.
f. The extended release dosage forms of quetiapine as contemplated herein may be prepared as per the following examples, but these examples do not limit the scope of the invention.

### Example 1

### Composition of Extended Release Coated Pellets (with Seal Coating)

| | | |
|---|---|---|
| S. No. | Ingredients | Weight (mg) |

Seal Coated Cores

| | | |
|---|---|---|
| 1. | Non-pareil seeds | 125 |
| 2. | Cellulose acetate | 22.75 |
| 3. | Diethyl phthalate | 2.25 |
| 4. | Dichloromethane | q.s. |
| 5. | Methanol | q.s. |

Drug Layering

| | | |
|---|---|---|
| 6. | Quetiapine hemifumarate | 461 |
| 7. | Hydroxypropylmethylcellulose | 50 |
| 8. | Talc | 15 |
| 9. | Isopropyl alcohol | q.s. |
| 10. | Water | q.s. |

Rate-controlling Coating

| | | |
|---|---|---|
| 11. | Eudragit ® RS 30D | 64.4 |
| 12. | Eudragit ® RL 30D | 16.1 |
| 13. | Talc | 40.25 |
| 14. | Triethylcitrate | 16.1 |
| 15. | Purified Water | q.s. |

### Procedure:

1) Cellulose acetate was dispersed in dichloromethane followed by addition of methanol and diethyl phthalate.
2) Non-pareil seeds were loaded in Glatt coater and seal coated with the above dispersion of step 1.
3) Hydroxypropylmethylcellulose was dispersed in isopropyl alcohol and water.
4) Milled quetiapine hemifumarate was added to dispersion of step 3 and stirred for 20 minutes. To this, talc was added and stirred for 15 minutes.
5) Seal coated non-pareil seeds of step 2 were loaded in Glatt coater and coated with drug layer using dispersion of step 4.
6) The above drug layered pellets of step 5 were coated in Glatt coater with the coating composition containing talc, triethyl citrate and Eudragit® dispersed in purified water.

The cores with varying percentages of rate-controlling coating were prepared. The coated pellets of Example I were subjected to dissolution studies in USP I dissolution apparatus in 900 mL phosphate buffer of pH 6.8 at 100 rpm. The results are given in Table 1.

TABLE 1
Dissolution Profile of Coated Pellets of Example 1 with Varying
Amounts of Rate-Controlling Coating in USP I Dissolution Apparatus
in 900 mL Phosphate Buffer of pH 6.8 at 100 rpm

| | |
|---|---|
| | Percentage of drug released for specific |
| Time | amounts of rate-controlling coating |

| (hours) | 15.1% | 20.4% |
|---|---|---|
| 0 | 0 | 0 |
| 1 | 22 | 17 |
| 2 | 35 | 27 |
| 4 | 49 | 40 |
| 6 | 59 | 50 |
| 8 | 66 | 57 |
| 12 | 75 | 67 |
| 16 | 81 | 75 |
| 20 | 84 | 79 |
| 24 | 84 | 82 |

### Examples 2-3

### Composition of Extended Release Coated Pellets (without Seal Coat)

| | |
|---|---|
| Example 2 | Example 3 |

| | | |
|---|---|---|
| S. No. | Ingredients | Weight (mg) |

Drug Layering

| | | | |
|---|---|---|---|
| 1. | Non-pareil seeds | 145 | 145 |
| 2. | Quetiapine hemifumarate | 461 | 461 |
| 3. | Hydroxypropylmethylcellulose | 50 | 50 |
| 4. | Talc | 15 | 15 |
| 5. | Isopropyl alcohol | q.s. | q.s. |
| 6. | Purified water | q.s. | q.s. |

Rate-controlling Coating

| | | | |
|---|---|---|---|
| 7. | Eudragit ® RS30D | 80.42 | 79.90 |
| 8. | Eudragit ® RL30D | 20.10 | — |
| 9. | Talc | 50.26 | 39.90 |
| 10. | Triethyl citrate | 20.10 | 15.99 |
| 11. | Purified water | q.s. | q.s. |

### Procedure:

1) Dispersed hypromellose and talc in isopropyl alcohol. To this, water was added and quetiapine hemifumarate was dispersed in it.
2) Non-pareil seeds were loaded in Glatt coater and coated with dispersion of step 1.
3) The above drug layered cores were coated in Glatt coater with the coating composition containing talc, triethyl citrate and Eudragit® dispersed in purified water.

The cores with varying percentages of rate-controlling coating were prepared. The coated pellets of Example 2 were subjected to dissolution studies in USP I dissolution apparatus in 900 mL phosphate buffer of pH 6.8 at 100 rpm. The results are given in Table 2.

TABLE 2
Dissolution Profile of Coated Pellets of Example 2 with Varying
Amounts of Rate-controlling Coating in USP I Dissolution Apparatus
in 900 mL Phosphate Buffer of pH 6.8 at 100 rpm

| | Percentage of drug released for specific amounts of rate- | | | | |
|---|---|---|---|---|---|
| Time | controlling coating | | | | |
| (hours) | 5.7% | 9.7% | 16.1% | 22.5% | 25.5% |
| 0 | 0 | 0 | 0 | 0 | 0 |
| 1 | 18 | 17 | 14 | 13 | 13 |
| 2 | 30 | 26 | 23 | 22 | 20 |
| 4 | 46 | 39 | 31 | 29 | 29 |
| 6 | 57 | 48 | 39 | 36 | 36 |
| 8 | 66 | 55 | 45 | 42 | 43 |
| 12 | 78 | 66 | 55 | 51 | 50 |
| 16 | 83 | 72 | 64 | 59 | 60 |
| 20 | 86 | 78 | 72 | 66 | 66 |
| 24 | 87 | 81 | 77 | 75 | 73 |

The coated pellets of Example 3 were subjected to dissolution studies in USP I dissolution apparatus in 900 mL of 0.1N HCl at 100 rpm. The results are given in Table 3.

TABLE 3
Dissolution Profile of Coated Pellets of Example 3 with
Varying Amounts of Rate-controlling Coating in USP I Dissolution
Apparatus in 900 mL of 0.1N HCl at 100 rpm

| | |
|---|---|
| | Percentage of drug released for specific |
| Time | amounts of rate-controlling coating |

| (hours) | 11.6% | 20.2% |
|---|---|---|
| 0 | 0 | 0 |
| 1 | 60 | 27 |
| 2 | 76 | 40 |
| 4 | 95 | 64 |
| 6 | 99 | 79 |
| 8 | 99 | 91 |
| 12 | 99 | 98 |
| 16 | 99 | 99 |

### Examples 4 and 5

### Composition of Tablets Containing Extended Release Coated Pellets of the Above Examples 2 and 3

| | | Example 4 | Example 5 |
|---|---|---|---|
| | | (Tablets containing | (Tablets containing |
| | | extended release coated | extended release coated |
| | | pellets of Example 2) | pellets of Example 3) |
| S. No. | Ingredients | Weight (mg) | |
| 1. | Extended release coated pellets | 839.5 | 806.8 |
| 2. | Microcrystalline cellulose | 261.2 | 284.9 |
| 3. | Polyethylene glycol | 53.6 | 58.5 |
| 4 | Croscarmellose sodium | 43.3 | 47.3 |
| 5. | Magnesium stearate | 1.6 | 1.8 |
| 6. | Colloidal silicon dioxide | 0.6 | 0.7 |

### Procedure:

1) Extended release coated pellets were mixed with microcrystalline cellulose, polyethylene glycol and croscarmellose sodium.
2) Blend of step 1 was lubricated with magnesium stearate and colloidal silicon dioxide and then compressed into tablets.

Above tablets of Examples 4 and 5 were subjected to dissolution studies in USP I dissolution apparatus at 100 rpm in 900 mL of (a) 0N HCl and (b) phosphate buffer of pH 6.8. The results are given in Tables 4 and 5 respectively.

TABLE 4
Dissolution Profile of Tablets of Example 4 in USP
I Dissolution Apparatus at 100 rpm in 900 mL of (a)
0.1N HCl and (b) Phosphate Buffer of pH 6.8

| | |
|---|---|
| Time | Percentage of drug released |

| (hours) | 0.1N HCl | Phosphate buffer (pH 6.8) |
|---|---|---|
| 0 | 0 | 0 |
| 1 | 93 | 11 |
| 2 | 102 | 17 |
| 4 | 103 | 25 |
| 6 | 104 | 31 |
| 8 | 103 | 36 |
| 12 | 103 | 44 |

TABLE 5
Dissolution Profile of Tablets of Example 5 in USP
I Dissolution Apparatus at 100 rpm in 900 mL of (a)
0.1N HCl and (b) Phosphate Buffer of pH 6.8

| | |
|---|---|
| Time | Percentage of drug released |

| (hours) | 0.1N HCl | Phosphate buffer (pH 6.8) |
|---|---|---|
| 0 | 0 | 0 |
| 1 | 56 | 8 |
| 2 | 73 | 14 |
| 4 | 87 | 22 |
| 6 | 92 | 27 |
| 8 | 95 | 32 |
| 12 | 97 | 39 |

The multiple unit extended release dosage form may also be prepared as per the following examples:

### Examples 6-7

### Composition of Quetiapine Tablets Containing Extended Release Coated Cores

| | |
|---|---|
| Example 6 | Example 7 |

| | | |
|---|---|---|
| S. No. | Ingredients | Weight (mg) |

Drug Layering

| | | | |
|---|---|---|---|
| 1. | Quetiapine hemifumarate | 461.0 | 461.0 |
| 2. | Non-pareil sugar seeds | 100.0 | — |
| 3. | Microcrystalline spheres | — | 100.0 |
| 4. | Hydroxypropylmethylcellulose | 19.0 | 19.0 |
| 5. | Talc | 30.0 | 30.0 |
| 6. | Isopropyl alcohol | q.s. | q.s. |
| 7. | Purified water | q.s. | q.s. |

Extended Release Coating

| | | | |
|---|---|---|---|
| 8. | Eudragit ® RS30D | 59.42 | 59.42 |
| 9. | Eudragit ® RL30D | 5.94 | 5.94 |
| 10. | Triethyl citrate | 6.54 | 6.54 |
| 11. | Talc | 19.6 | 19.6 |
| 12. | Purified water | q.s. | q.s. |

Sub-coating

| | | | |
|---|---|---|---|
| 13. | Polyethylene glycol | 70.1 | 70.1 |
| 14. | Isopropyl alcohol | q.s. | q.s. |
| 15. | Purified water | q.s. | q.s. |

Tableting Ingredients

| | | | |
|---|---|---|---|
| 16. | Microcrystalline cellulose | 115.9 | 115.9 |
| 17. | Croscarmellose sodium | 25 | 25 |
| 18. | Polyethylene glycol | 80 | 80 |
| 19. | Magnesium stearate | 10 | 10 |
| 20. | Talc | 7.5 | 7.5 |
| 21. | Opadry ® (film coat) | 30.0 | 30.0 |

### Procedure:

1) Disperse hydroxypropylmethylcellulose and talc in isopropyl alcohol/water and disperse quetiapine hemifumarate in it.
2) Load the non-pareil sugar seeds (Example 6) or microcrystalline cellulose spheres (Example 7) in Glatt coater and coat with drug layer using dispersion of step 1.
3) Disperse talc, triethyl citrate in purified water and add Eudragit® dispersion to it.
4) Load the drug layered beads in Glatt coater and coat using Eudragit® dispersion to desired weight gain.
5) Prepare the solution of polyethylene glycol in isopropyl alcohol: water solvent mixture and coat extended release pellets using this solution.
6) Mix polyethylene glycol coated pellets with extragranular ingredients and compress into tablets.
7) Film coat the tablets using Opadry®.

### Examples 8-9

### Composition of Quetiapine Tablets Containing Extended Release Coated Cores

| | |
|---|---|
| Example 8 | Example 9 |

| | | |
|---|---|---|
| S. No. | Ingredients | Weight (mg) |

Drug Layering

| | | | |
|---|---|---|---|
| 1. | Quetiapine hemifumarate | 461.0 | 461.0 |
| 2. | Non-pareil sugar seeds | 100 | — |
| 3. | Microcrystalline spheres | — | 100 |
| 4. | Hydroxypropylmethylcellulose | 19.0 | 19.0 |
| 5. | Talc | 30.0 | 30.0 |
| 6. | Isopropyl alcohol | q.s. | q.s. |
| 7. | Purified water | q.s. | q.s. |

Extended Release Coating

| | | | |
|---|---|---|---|
| 8. | Cellulose acetate | 40.67 | 40.67 |
| 9. | Diethyl phthalate | 8.13 | 8.13 |
| 10. | Acetone | q.s. | q.s. |
| 11. | Purified water | q.s. | q.s. |

Sub-coating

| | | | |
|---|---|---|---|
| 12. | Polyethylene glycol | 65.88 | 65.88 |
| 13. | Isopropyl alcohol | q.s. | q.s. |
| 14. | Purified water | q.s. | q.s. |

Tableting Ingredients

| | | | |
|---|---|---|---|
| 15. | Microcrystalline cellulose | 116.32 | 116.32 |
| 16. | Croscarmellose sodium | 25 | 25 |
| 17. | Polyethylene glycol | 80 | 80 |
| 18. | Magnesium stearate | 8 | 8 |
| 19. | Talc | 6 | 6 |
| 20. | Opadry ® (film coat) | 30.0 | 30.0 |

### Procedure:

1) Disperse hydroxypropylmethylcellulose and talc in isopropyl alcohol/water and disperse quetiapine hemifumarate in it.
2) Load the non-pareil sugar seeds (Example 8) or microcrystalline cellulose spheres (Example 9) in Glatt coater and coat with drug layer using dispersion of step 1.
3) Dissolve cellulose acetate and diethyl phthalate in mixture of acetone and water.
4) Load the drug layered beads in Glatt coater and coat using cellulose acetate solution to desired weight gain.
5) Prepare the solution of polyethylene glycol in isopropyl alcohol: water solvent mixture and coat extended release pellets using this solution.
6) Mix polyethylene glycol coated pellets with extragranular ingredients and compress into tablets.
7) Film coat the tablets using Opadry®.

### Examples 10-11

### Composition of Quetiapine Tablets Containing Extended Release Coated Cores

| | |
|---|---|
| Example 10 | Example 11 |

| | | |
|---|---|---|
| S. No. | Ingredients | Weight (mg) |

Drug Layering

| | | | |
|---|---|---|---|
| 1. | Quetiapine hemifumarate | 461.0 | 461.0 |
| 2. | Non-pareil sugar seeds | 100 | — |
| 3. | Microcrystalline spheres | — | 100 |
| 4. | Hydroxypropylmethylcellulose | 19.0 | 19.0 |
| 5. | Talc | 30.0 | 30.0 |
| 6. | Isopropyl alcohol | q.s. | q.s. |
| 7. | Purified water | q.s. | q.s. |

Extended Release Coating

| | | | |
|---|---|---|---|
| 8. | Ethyl cellulose | 101.67 | 101.67 |
| 9. | Diethyl phthalate | 20.33 | 20.33 |
| 10. | Acetone | q.s. | q.s. |
| 11. | Isopropyl alcohol | q.s. | q.s. |

Sub-coating

| | | | |
|---|---|---|---|
| 12. | Polyethylene glycol | 73.2 | 73.2 |
| 13. | Isopropyl alcohol | q.s. | q.s. |
| 14. | Purified water | q.s. | q.s. |

Tableting Ingredients

| | | | |
|---|---|---|---|
| 15. | Microcrystalline cellulose | 115.8 | 115.8 |
| 16. | Croscarmellose sodium | 25 | 25 |
| 17. | Polyethylene glycol | 80 | 80 |
| 18. | Magnesium stearate | 8 | 8 |
| 19. | Talc | 6 | 6 |
| 20. | Opadry ® (film coat) | 30.0 | 30.0 |

### Procedure:

1) Disperse hydroxypropylmethylcellulose and talc in isopropyl alcohol/water and disperse quetiapine hemifumarate in it.
2) Load the non-pareil sugar seeds (Example 10) or microcrystalline cellulose spheres (Example 11) in Glatt coater and coat with drug layer using dispersion of step 1.
3) Dissolve ethyl cellulose and diethyl phthalate in mixture of acetone and isopropyl alcohol.
4) Load the drug layered beads in Glatt coater and coat using ethyl cellulose solution to desired weight gain.
5) Prepare the solution of polyethylene glycol in isopropyl alcohol: water solvent mixture and coat extended release pellets using this solution.
6) Mix polyethylene glycol coated pellets with extragranular ingredients and compress into tablets.
7) Film coat the tablets using Opadry®.

We claim:

1. A multiple unit extended release dosage form of quetiapine, wherein each unit comprises:
a. an inert core coated with a solution/dispersion comprising quetiapine and one or more pharmaceutically acceptable excipients; and
b. a rate-controlling coating comprising one or more of ammoniomethacrylate copolymer, methacrylic acid copolymer, cellulose acetate, ethyl cellulose and one or more pharmaceutically acceptable excipients.

2. A multiple unit extended release dosage form of quetiapine according to claim 1, wherein the rate-controlling coating comprising ammoniomethacrylate copolymer.

3. A multiple unit extended release dosage form of quetiapine according to claims 1 or 2, wherein pharmaceutically acceptable excipients comprise one or more of plasticizers, solvents, binders, diluents, disintegrants, pH modifiers, lubricants, glidants, or mixtures thereof.

4. A process for the preparation of multiple unit extended release dosage form of quetiapine according to claim 1, the process comprising the steps of:
a. Optionally, coating the inert cores with a seal coat comprising a solution/dispersion of the polymer;
b. Applying a solution/dispersion of quetiapine and one or more pharmaceutically acceptable excipients in a suitable solvent onto the inert cores; and
c. Applying a solution/dispersion of rate-controlling polymer comprising one or more of ammoniomethacrylate copolymer, methacrylic acid copolymer, cellulose acetate, ethyl cellulose and one or more pharmaceutically acceptable excipients on the drug layered cores.

5. A multiple unit extended release dosage form of quetiapine according to claim 1, wherein the multiple units exhibit the following dissolution profile when measured in USP Type I dissolution apparatus in 900 ml of phosphate buffer (pH 6.8) at 100 rpm:
at most 50% of the drug is released in 2 hours;
at most 70% of the drug is released in 6 hours; and
at most 90% of the drug is released in 12 hours.

6. A multiple unit extended release dosage form of quetiapine according to claim 1, wherein the multiple units are compressed into tablets.

7. A multiple unit extended release dosage form of quetiapine according to claim 1, wherein the multiple units are filled into capsules or sachets.
